(19) United States
(12) Patent Application Publication
(10) Pub. No.: US 2022/0380721 A1
(43) Pub. Date: Dec. 1, 2022

(54) METHOD FOR COUPLING ANTIBODY TO SURFACE OF CELL AND METHOD FOR APPLYING CELL COUPLED WITH THE ANTIBODY

(71) Applicant: Xiamen Nuokangde Biological Technology Co., Ltd., Xiamen (CN)

(72) Inventors: Zhu LI, Xiamen (CN); Pinghong LIAN, Xiamen (CN); Xianwu WANG, Xiamen (CN)

(21) Appl. No.: 17/828,811

(22) Filed: May 31, 2022

Related U.S. Application Data
(63) Continuation of application No. PCT/CN2020/132028, filed on Nov. 27, 2020.

(30) Foreign Application Priority Data
Nov. 28, 2019 (CN) 201911196567.X

(51) Int. Cl. C12N 5/00; C12N 5/078; C12N 5/0783

(52) U.S. Cl. CPC C12N 5/0006; C12N 5/0634; C12N 5/0646

(57) ABSTRACT

A method for coupling an antibody to a surface of a cell comprises the following steps: (1) chemically modifying sialic acid to obtain a sialic acid derivative containing an azide group; (2) absorbing the sialic acid derivative by the cell to obtain a cell modified with the azide group; (3) modifying the antibody with a conjunction compound to obtain a modified antibody; (4) co-culturing the modified antibody with the cell modified with the azide group. The present disclosure modifies natural sialic acid molecules in vitro through chemical synthesis methods, and utilizes modified natural sialic acid molecules to realize antibody modification on the surface of the cell. The modification method of the present disclosure is simple, low-cost, safe, and efficient, does not need complex gene editing or enzyme catalytic operation, and has universality. In theory, the modification method can realize the coupling of any antibody or macro-molecular substance on the surface of the cell.

### RELATED APPLICATIONS

This application is a continuation of and claims priority to International patent application number PCT/CN2020/132028, filed Nov. 27, 2020, which claims priority to Chinese patent application number 201911196567.X, filed on Nov. 28, 2019. International patent application number PCT/CN2020/132028 and Chinese patent application number 201911196567.X are incorporated herein by reference.

### FIELD OF THE DISCLOSURE

The present disclosure relates to bio-engineering, and in particular relates to a method for coupling an antibody to a surface of a cell and a method for applying a cell coupled with the antibody.

### BACKGROUND OF THE DISCLOSURE

As tumor treatment enters the era of cellular immunotherapy, people have made major breakthroughs in the study of fighting cancer. The current mainstream tumor immunotherapy can be divided into adoptive cell therapy and immune checkpoint inhibitor therapy. The former can be divided into autologous cell therapy (cells from patients) and allogeneic cell therapy (cells from third-party donors) according to the cell source. Generally, immune cells are extracted from patients or other human beings, transformed and amplified, and then returned to patients to fight tumors. CAR-T (Chimeric Antigen Receptor T-cell) therapy is the leader of this type of therapy and has received extensive attention and research. Other immune checkpoint inhibitory therapies, such as PD-1 antibody, PD-L1 antibody, CTLA-4 antibody, etc., are based on the principle of relieving the tumor's tolerance to immune cells and restoring the recognition and killing function of immune cells to tumors, thereby achieving the purpose of fighting cancer. Today, with the continuous investment of international pharmaceutical giants in cellular immunotherapy, related technologies will continue to make breakthroughs, and the cell therapy market has a bright future. But at the same time, it also faces many challenges, such as cell cytokine storm and off-target. At the same time, CAR-T is complicated and difficult to operate in the production process, and the resulting high treatment costs are also unbearable for ordinary families. PD 1 immune checkpoint blockade therapy has drug resistance and low efficacy, coupled with high treatment costs, and therefore continuous technological breakthroughs and innovations are still needed to overcome these difficulties.

### BRIEF SUMMARY OF THE DISCLOSURE

An objective of the present disclosure is to solve the deficiencies of the existing techniques and provide a method for coupling an antibody to a surface of a cell and a method for applying the cell coupled with the antibody.

A technical solution of the present disclosure is as follows.

A method for coupling an antibody to a surface of a cell, comprising:

(1) chemically modifying sialic acid to obtain a sialic acid derivative containing an azide group;

(2) absorbing the sialic acid derivative by the cell, and expressing the azide group on a membrane surface of the cell to obtain a cell modified with the azide group through a sialic acid metabolic pathway of the cell;

(3) modifying the antibody with a conjunction compound to obtain a modified antibody, wherein a first end of the conjunction compound has a first active group configured to react with a sulfhydryl group or an amino group, and a second end of the conjunction compound has a second active group configured to bio-orthogonally react with the azide group, and the first active group is connected to the sulfhydryl group or the amino group of the antibody by a first reaction; and

(4) co-culturing the modified antibody with the cell modified with the azide group to enable the second active group of the conjunction compound of the modified antibody to be connected to the azide group of the cell modified with the azide group by a second reaction.

In a preferred embodiment of the present disclosure, the sialic acid derivative containing the azide group comprises a compound having any one of the following structural formulas:

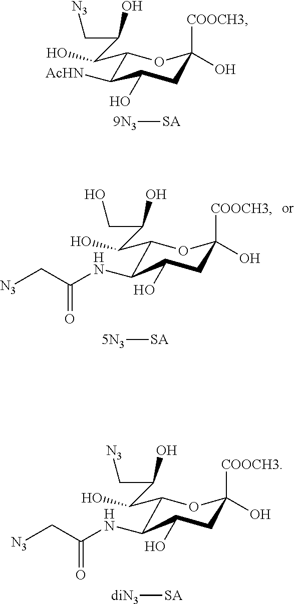

In a preferred embodiment of the present disclosure, the first active group comprises a succinimide active ester group or a maleimide group.

In a preferred embodiment of the present disclosure, the conjunction compound comprises a compound having any one of the following structural formulas:

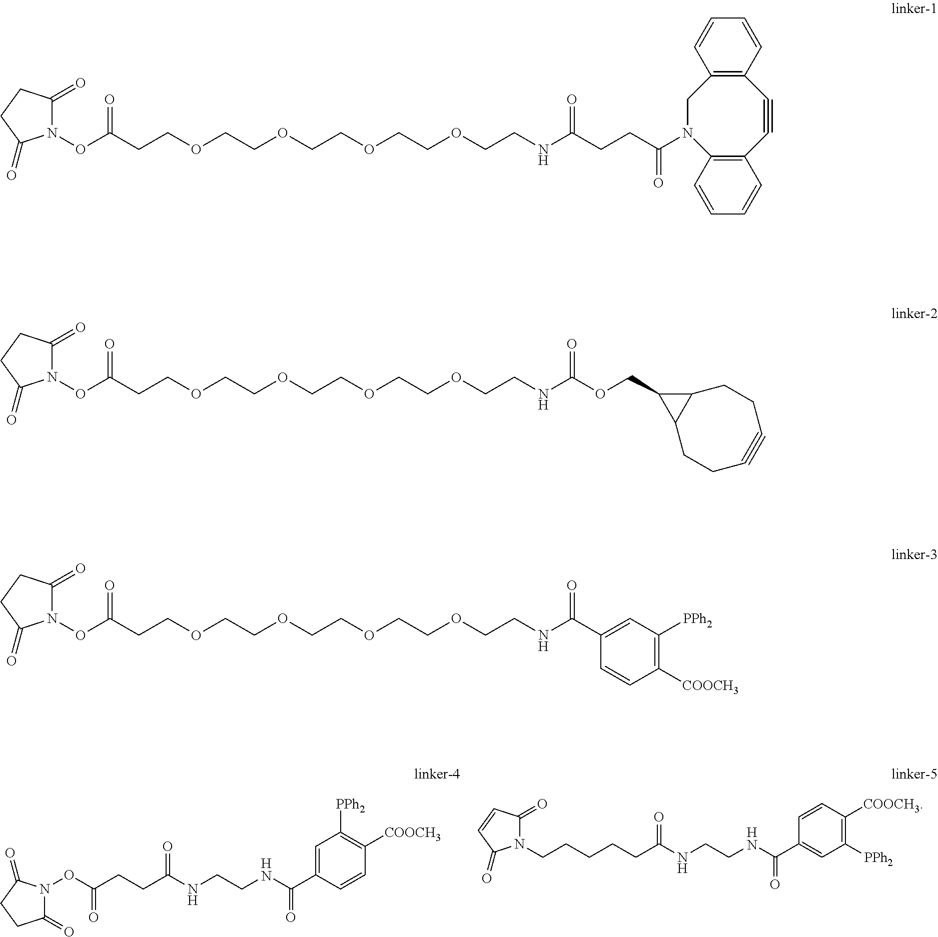

In a preferred embodiment of the present disclosure, the cell is an immune cell.

In a preferred embodiment of the present disclosure, the cell is an original human T cell or a natural killer cell.

A cell, a surface of the cell comprises an azide group expressed on the surface by absorbing a sialic acid derivative containing the azide group through a sialic acid metabolic pathway, the azide group is connected to an antibody through a conjunction compound, a first end of the conjunction compound has a first active group configured to react with a sulfhydryl group or an amino group, a second end of the conjunction compound has a second active group configured to react with the azide group by a bio-orthogonal reaction, the first active group is connected to the sulfhydryl group or the amino group of the antibody by a first reaction, and the second active group is connected to the azide group by a second reaction.

In a preferred embodiment of the present disclosure, the sialic acid derivative containing the azide group comprises a compound having any one of the following structural formulas:

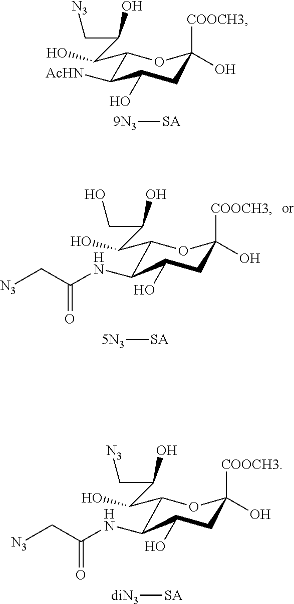

In a preferred embodiment of the present disclosure, the first active group comprises a succinimide active ester group or a maleimide group.

In a preferred embodiment of the present disclosure, the conjunction compound comprises a compound having any one of the following structural formulas:

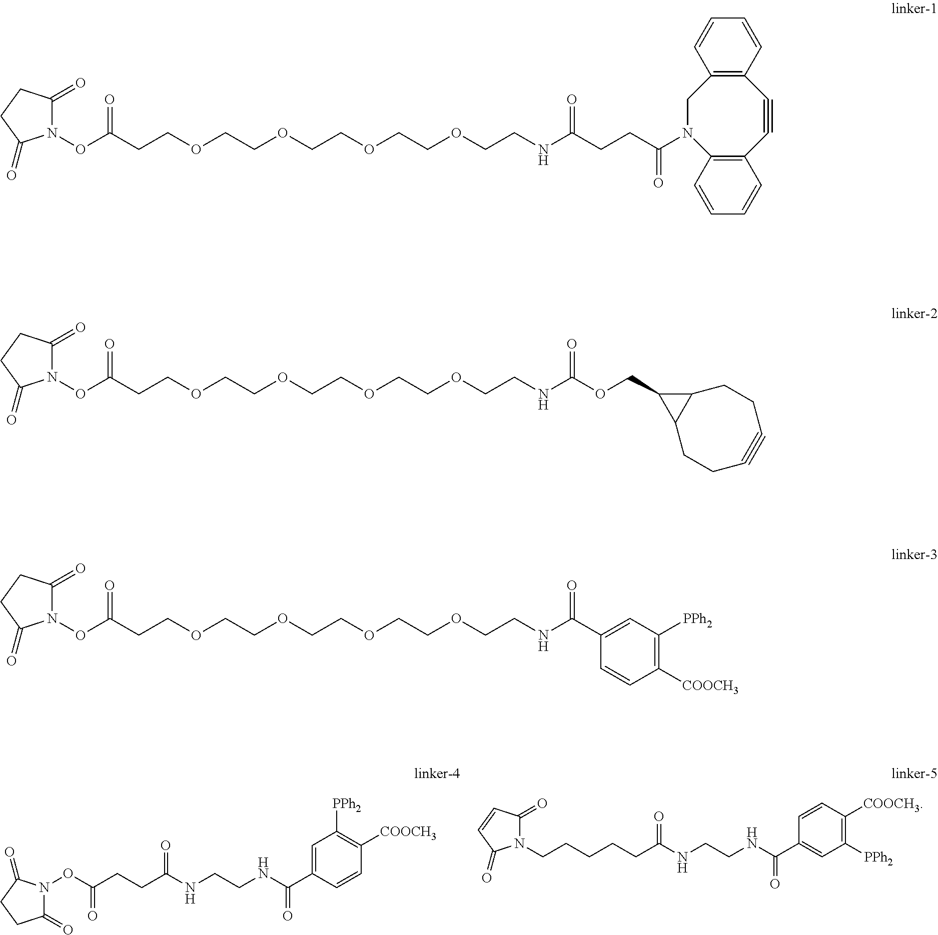

In a preferred embodiment of the present disclosure, the cell is an immune cell.

The present disclosure has the following advantages.

1. The present disclosure modifies natural sialic acid molecules in vitro through chemical synthesis methods, and utilizes modified natural sialic acid molecules to realize antibody modification on the surface of the cell.

2. The modification method of the present disclosure is simple, low-cost, safe, and efficient, does not need complex gene editing or enzyme catalytic operation, and has universality. In theory, the modification method can realize the coupling of any antibody or macro-molecular substance on the surface of the cell.

### BRIEF DESCRIPTION OF THE DRAWINGS

FIG. 1 illustrates a principle of the present disclosure.

FIG. 2 illustrates principles of a connection between an antibody and a conjunction compound and a connection between the antibody and a cell through a bio-orthogonal reaction of the present disclosure.

FIG. 3 illustrates a principle that Fluorescein-Stau (FITC-Stau) detects azide groups on a cell surface of embodiment 1 of the present disclosure.

FIG. 4 illustrates a fluorescence image of FITC-Stau according to an azido sialic acid on a surface of the NK92 cells of embodiment 1 of the present disclosure.

FIG. 5 illustrates the surface of NK92 cells coupled with anti-Her2 detected by a flow cytometry of embodiment 1 of the present disclosure.

FIG. 6 illustrates a killing state of SK-BR-3 cells overexpressing Her2 using the NK92 cells coupled with the anti-Her2 of embodiment 1 of the present disclosure.

FIG. 7 illustrates a graph of therapeutic effects of breast cancer in mice using the NK92 cells coupled with the anti-Her2 of embodiment 1 of the present disclosure.

FIG. 8 illustrates a flow cytometry result of the NK92 cells coupled with human IgG antibody of embodiment 1 of the present disclosure.

FIG. 9 illustrates a flow cytometry result of the NK92 cells coupled with murine IgG antibody of embodiment 1 of the present disclosure.

FIG. 10 illustrates a flow cytometry result of the NK92 cells coupled with rabbit IgG antibody of embodiment 1 of the present disclosure.

FIG. 11 illustrates a confocal laser image of the NK92 cells coupled with multiple antibodies from different species of embodiment 1 of the present disclosure.

FIG. 12 illustrates a retention time of a coupled cetuximab, a monoclonal antibody, on the surface of the NK92 cells of embodiment 1 of the present disclosure.

FIG. 13 illustrates killing results in vitro of SW480 cells using the NK92 cells coupled with cetuximab, the monoclonal antibody, of embodiment 1 of the present disclosure.

FIGS. 14A, 14B, 14C, 14D, and 14E illustrate anti-tumor results in mice in vivo of the NK92 cells coupled with cetuximab, the monoclonal antibody, of embodiment 1 of the present disclosure.

### DETAILED DESCRIPTION OF THE EMBODIMENTS

The present disclosure will be further described below in combination with the accompanying drawings and embodiments.

### Embodiment 1

1. A Mechanism of a Coupling Reaction

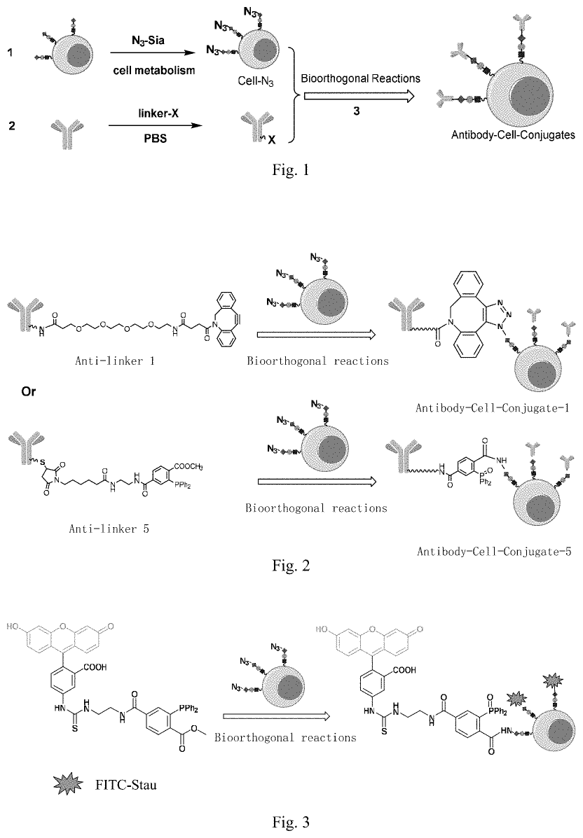

Referring to FIG. 1, sialic acid derivatives modified with an azide group are added during a cell growth process to obtain cells modified with the azide group (Cell-N3) on a surface using a cellular sialic acid metabolism pathway. A modification of antibody is based on a reaction between a remaining amino group or a remaining sulfhydryl group on an amino acid residue chain of an antibody protein and a succinimide active ester group or a maleimide group in a conjunction compound, linker-x, and the other end of the conjunction compound comprises a ligand group configured to have a biological orthogonal reaction with the azide group. A purpose of cells coupled with antibody using a covalent bond is complete.

A preferred structural formula of the sialic acid derivatives modified with the azide group is as follows:

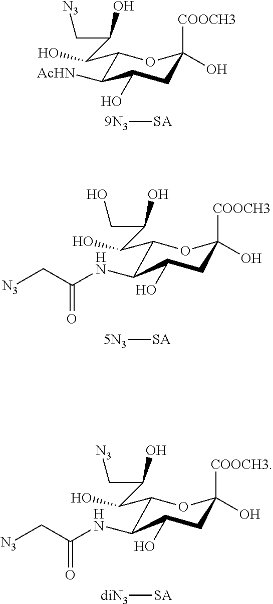

A preferred structural formula of the conjunction compound is as follows:

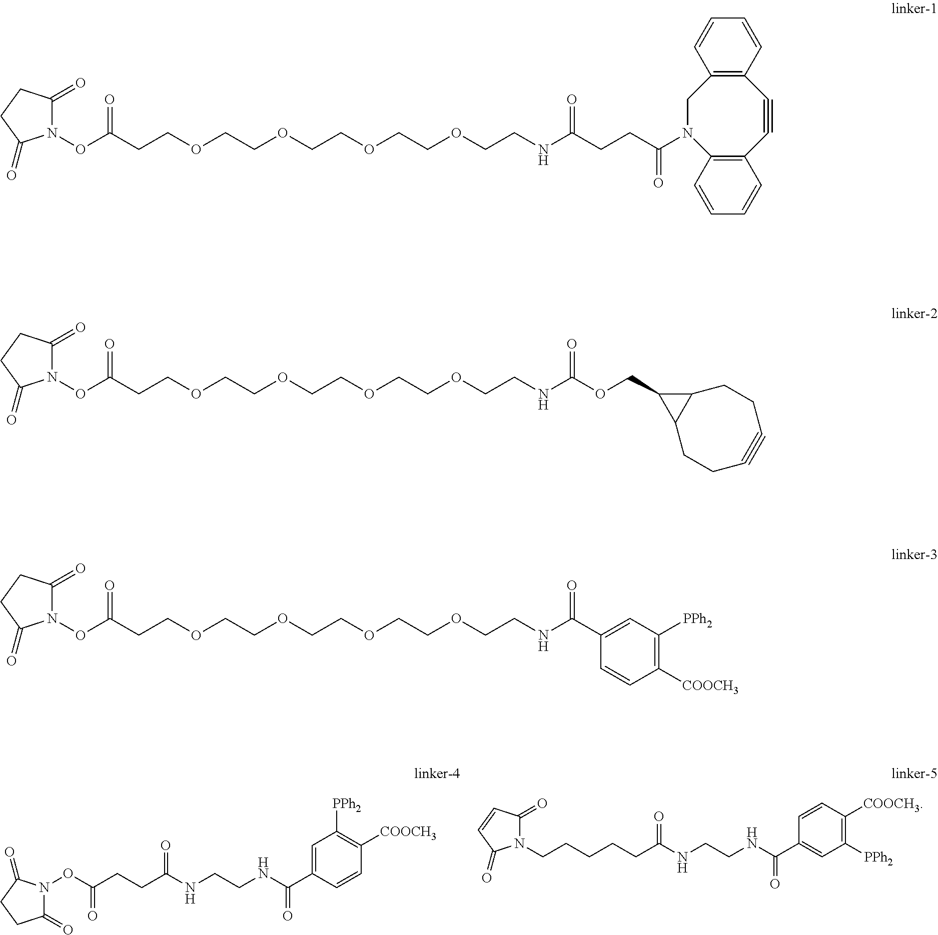

2. Steps for Synthesizing Related Molecules

9N₃-SA is synthesized according to the literature (Cheng B, et al. ACS chemical biology, 2019, 14, 2141, which is incorporated herein by reference). Synthesis methods of 5N₃-SA and diN₃-SA are as follows:

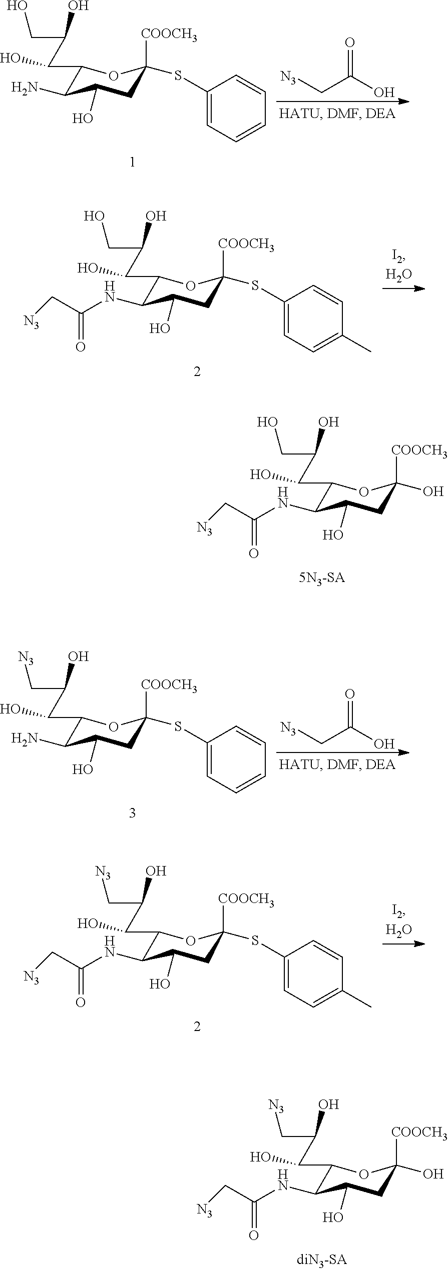

A Synthesis of 5N₃-SA

1) 500 mg of a compound 1 (the compound 1 is synthesized step according to the literature: Abdu-Allah, et al. Journal of Medicinal Chemistry, 2008, 51, 6665, which is incorporated herein by reference) is dissolved in 10 mL of N,N-Dimethylformamide (DMF), then 736 mg of (2-(7-Azabenzotriazol-1-yl)-N,N,N′,N′-tetramethyluronium hexafluorophosphate) (HATU), 500 mg of N,N-Diisopropylethylamine (DIEA), and 156 mg of 2-azidoacetic acid are added to obtain a first reaction solution. The first reaction solution is stirred overnight at room temperature (e.g., 20-25° C.), and the first reaction solution is then concentrated and is purified with a silica gel column to obtain 300 mg of an intermediate 2.

2) 300 mg of the intermediate 2 is added in 30 mL of deionized water, 323 mg of I₂ is then added to obtain a second reaction solution, and the second reaction solution is stirred for 24 hours at room temperature. The second reaction solution is then extracted, concentrated, and purified using a high pressure liquid chromatography to obtain 100 mg of the 5N3-SA.

A Synthesis of diN₃-SA

1) 500 mg of a compound 3 (the compound 3 is synthesized according to the literature: Peng W, Paulson J C. Journal of the American Chemical Society, 2017, 139, 12450, which is incorporated herein by reference) is dissolved in 10 mL of N,N-Dimethylformamide (DMF), then 691 mg of the HATU, 500 mg of DIEA, and 156 mg of 2-azidoacetic acid are added to obtain a third reaction solution. The third reaction solution is stirred overnight at room temperature, and the third reaction solution is then concentrated and is purified with a silica gel column to obtain 280 mg of an intermediate 4.

2) 280 mg of the intermediate 4 is dissolved in 30 mL of deionized water, and 323 mg I₂ is added to obtain a fourth reaction solution. The fourth reaction solution is stirred for 24 hours at room temperature. The fourth reaction solution is extracted, concentrated, and is purified using a high pressure liquid chromatography to obtain 80 mg of the diN₃-SA.

The conjunction compound can be purchased from some reagent websites (such as Chengdu Boerkang Biotechnology Co., LTD., Shanghai Bide Pharmaceutical Technology Co., LTD., etc.) or prepared by conventional organic synthesis methods.

For example, a route for synthesizing the linker-4 is as follows:

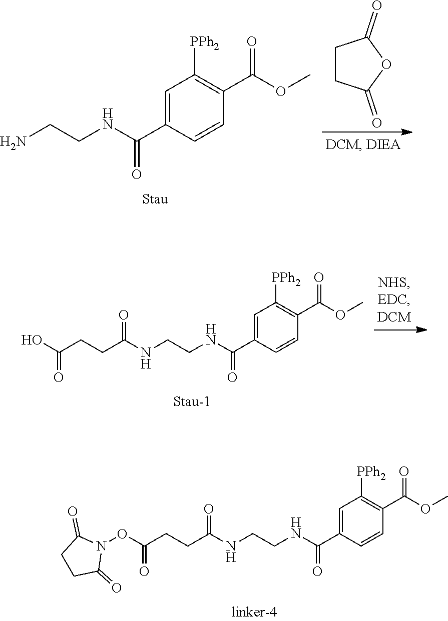

A preferred synthesis method is as follows: 500 mg of Stau is dissolved in 10 mL of dichloromethane, then 317 mg of DIEA, and 123 mg of succinic anhydride are added to obtain a fifth reaction solution, and the fifth reaction solution is stirred at room temperature for 1 hour. After the fifth reaction solution is concentrated, Stau-1 is obtained. The Stau-1 is directly ready for the next step without purification. 212 mg of N-hydroxysuccinimide (NHS), 354 mg of 1-(3-dimethylaminopropyl)-3-ethylcarbondiimide hydrochloride (EDC), and the Stau-1 are dissolved in 10 mL of dichloromethane, reacted at room temperature for 3 hours, and then extracted with ethyl acetate and water to obtain an organic phase. The organic phase is concentrated and purified by a silica gel column to obtain 120 mg of the Linker-4.

3. General Coupling Steps

1. Metabolic Incorporation of an Azido Analogues Sialic Acid in Cells

An appropriate amount of the azido analogues sialic acid is added into a normal cell culture medium with a final concentration of 100 μM (μMol/L). Cells are cultured in the normal cell culture medium under normal conditions for 24-48 hours to obtain azide modified cells (cell-N₃), and the azide modified cells are then washed with phosphate-buffered saline (PBS) for 2-3 times and replaced with another normal cell culture medium for later use.

2. Modification of the Antibody

The antibody is prepared into an antibody solution with a concentration of 6 mg/mL by PBS for use. 50 μL of a solution of the linker (the conjunction compound) prepared by dimethyl sulphoxide (DMSO) (a concentration of an original solution of the linker is 10 mM (mMol/L)) is added into 1 mL of the antibody solution and incubated at room temperature for 30 minutes. Then, 50 μL of a Tris buffer (pH 8.0, 1 M (Mol/L)) is added for quenching and quenched at room temperature for 5 minutes to obtain a solution of the IgG B antibody modified with the linker. The solution is ready for next cell-coupling steps.

3. Procedure for Coupling Cells with Antibody

100 μL of the solution of the IgG B antibody modified with the linker obtained in Step 2 is added into 1 mL of the cell-N₃ (a density of the cells is 1×10⁶ cells/mL) obtained in Step 1 and incubated at 37° C. for 2 hours. During this process, a bio-orthogonal reaction between an active group at a terminal of the linker of the IgG B antibody and the azide group on the cell surface is performed. The bio-orthogonal reaction can be complete under physiological conditions, and the IgG B antibody modified with the linker and the azide group on the cell surface does not react with the medium or other molecules in organisms; a principle is shown in FIG. 2. The cells coupled with the antibody are obtained by washing with PBS for 2-3 times.

4. Tumor Cells are Killed Using the Immune Cells with a Surface of the Immune Cells Coupled with the Antibodies

The present disclosure realizes the surface of the immune cells coupled with specific antibodies using the general coupling steps. In this embodiment, for example, a surface of NK92 cells coupled with antibodies of receptors (Her2) of breast cancer surface cells (anti-Her2 Ab) is used. The conjunction compound is Linker1, and the sialic acid derivative containing the azide group is 9N₃-SA.

1. Modification of the Antibodies

The anti-Her2 Ab is prepared into 6 mg/mL of an antibody solution by PBS, and 50 μL of linker1 original solution (10 mM) is added into 1 mL of the antibody solution and coupled at room temperature for 30 minutes. 50 μL of Tris buffer (pH 8.0, 1M) is then added and quenched at room temperature for 5 minutes to obtain a solution of the anti-Her2 Ab coupled with the linker1(anti-Her2-linker1), and the solution is stored at 4° C. for later use.

2. Metabolic Incorporation of the Azido Sialic Acid in NK92 Cells

NK92 cells normally cultured are distributed into a 24-well plate and then cultured in a NK92 cell medium containing 9N₃-SA with a final concentration of 100 μM for 24-48 hours. The NK92 cells are washed by PBS for 3 times, and the NK92 cell medium containing 9N₃-SA is then replaced by a normal NK92 medium to obtain NK92 cells modified with the azide groups. In order to verify that the NK92 cells have been modified with the azide groups, in this embodiment, Fluorescein-Stau (FITC-Stau) (as shown in FIG. 3), a ligand molecule connected to fluorescein and able to orthogonally react with the azide groups, is synthesized. The FITC-Stau is added into a medium of the NK92 cell modified with the azide groups with a final concentration of 100 μM, incubated at 37° C. for 2-4 hours, and washed with PBS for 3 times, and fluorescence on the cell surface is then observed by a confocal laser microscope. The results are shown in FIG. 4. Compared with cells of a control group, cells not modified with 9N₃-SA show no fluorescence on the cell surface, while cells modified with 9N₃-SA show strong fluorescence signal on the cell surface, which indicates that 9N₃-SA is absorbed by NK92 cells and metabolized to a terminal of glycoprotein.

A route for synthesizing FITC-Stau is as follows:

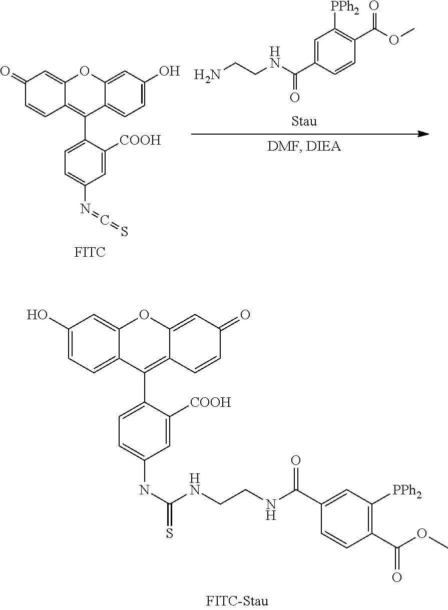

The FITC is a commercial product, the Stau is synthesized according to the literature (Chang, Pamela V., et al. Journal of the American Chemical Society. 2007, 129, 8400, which is incorporated herein by reference). A synthesis method is as follows: 200 mg of FITC is dissolved in 5 mL of DMF, 209 mg of the Stau and 200 mg of the DIEA are then added to obtain a sixth reaction solution, and the sixth reaction solution is stirred at room temperature for 5 hours. The sixth reaction solution is concentrated and then purified by a high pressure liquid chromatography to obtain 100 mg of FITC-Stau.

3. NK92 Cells with the Cell Surface Coupled with Her2 Antibody

100 μL of the solution of the anti-Her2-linker1 obtained in step 1 is added into 1 mL of the cells modified with the azide groups obtained in step 2 (a cell density is about 1×10⁶ cells/mL), incubated at 37° C. for 2-8 hours, and washed with PBS for 2-3 times to obtain the NK92 cells coupled with the Her2 antibody: anti-Her2-NK92 cells. In order to verify that the surface of the NK92 cells have been connected to the Her2 antibody, in this embodiment, the NK92 cells coupled with the Her2 antibody and the NK92 cells not coupled with antibodies are further treated by Allophycocyanin (APC)-anti-Fc, and a fluorescence signal of the APC is detected by a flow cytometry. Referring to FIG. 7, the fluorescence signal of the APC can be obviously observed on a surface of the NK92 cells coupled with the Her2 antibody, while the fluorescence signal cannot be observed on a surface of the NK92 cells not coupled with the antibodies in a control group, which indicates that the Her2 antibody is successfully coupled to the surface of the NK92 cells through the method of the present disclosure.

4. Killing Activity of the Anti-Her2-NK92 Cells Against SK-BR-3 Cells In Vitro

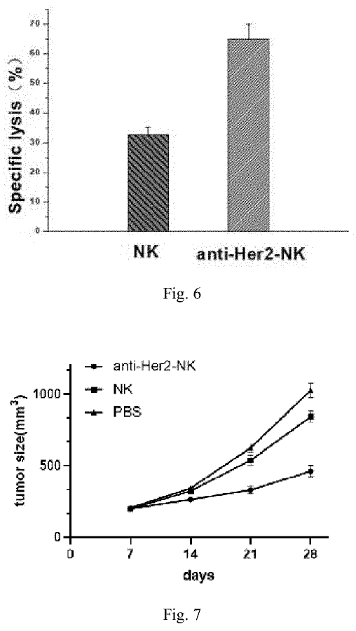

In this experiment, SK-BR-3 cells (Her2 protein is overexpressed, human breast cancer cells) are used as target cells to verify the killing activity of the anti-Her2-NK92 cells in vitro. The SK-BR-3 cells are mixed with the NK92 cells or the anti-Her2-NK92 cells in a ratio of 1:5 and then incubated in a 37° C. incubator for 4 hours. A release of lactate dehydrogenase in a supernatant is detected by a lactate dehydrogenase detection kit, and a cytotoxicity is calculated. The results are shown in FIG. 6.

5. Antitumor Activity of the Anti-Her2-NK92 Cells in Mice In Vivo

SK-BR-3 human breast cancer cells overexpressing Her2 are used to establish a subcutaneous tumor model of breast cancer in Balb/c nude mice in vivo, and the anti-Her2-NK92 cells (in an experimental group) are injected via a tail vein once a week from a seventh day after tumor inoculation (a tumor volume in the mice is about 200 mm³), and the number of cells is 10⁷ each time. The mice in a control group are injected with original NK92 cells, and the mice in a blank group are injected with a PBS solution at the same volume for three continuous weeks, and changes of the tumor volume are recorded. Referring to FIG. 7, the tumor volume of the mice injected with the anti-Her2-NK92 cells in the experimental group is significantly smaller than the tumor volume of the mice in the control group and the blank group, which indicates that the NK92 cells coupled with the Her2 antibody can enhance an activity of the NK92 cells for killing tumor cells to be used in cancer treatment.

5. Coupling Different Antibodies on a Same Cell Surface

The technology of the present disclosure differs from chimeric antibodies on the cell surface using gene editing. Different species of antibodies are easily coupled to the same cell surface using this technology.

5.1 Coupling Different Species of Antibodies on the Surface of the NK92 Cells.

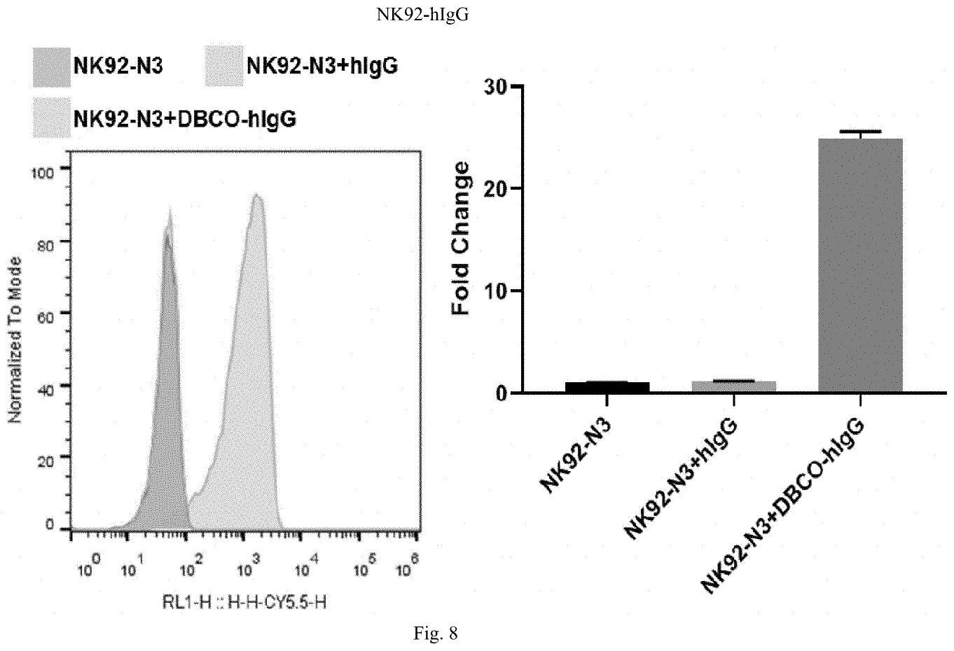

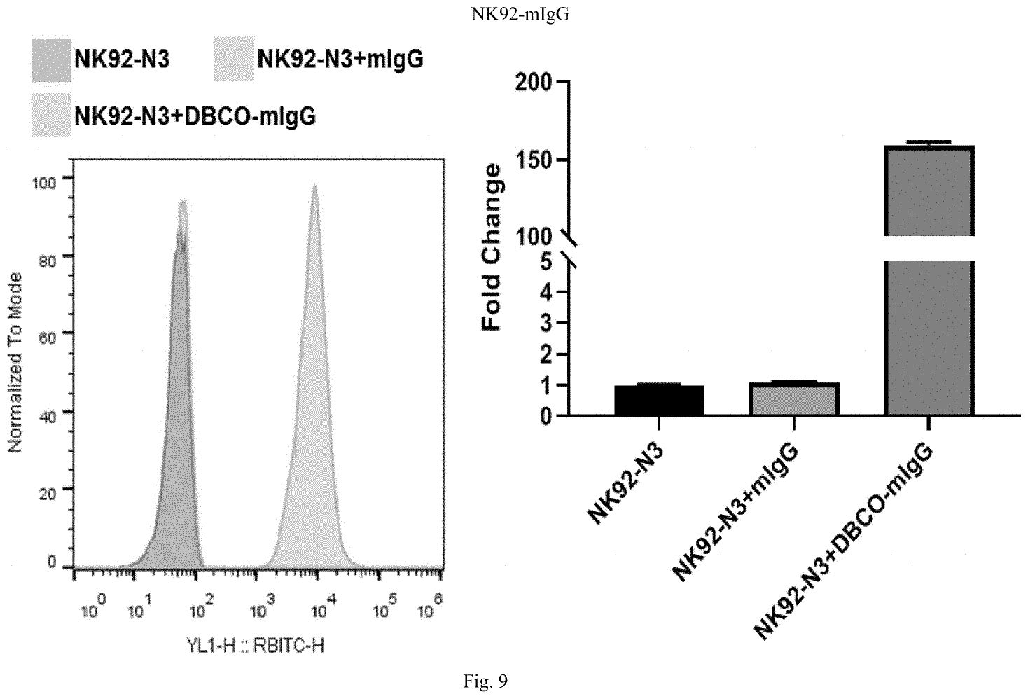

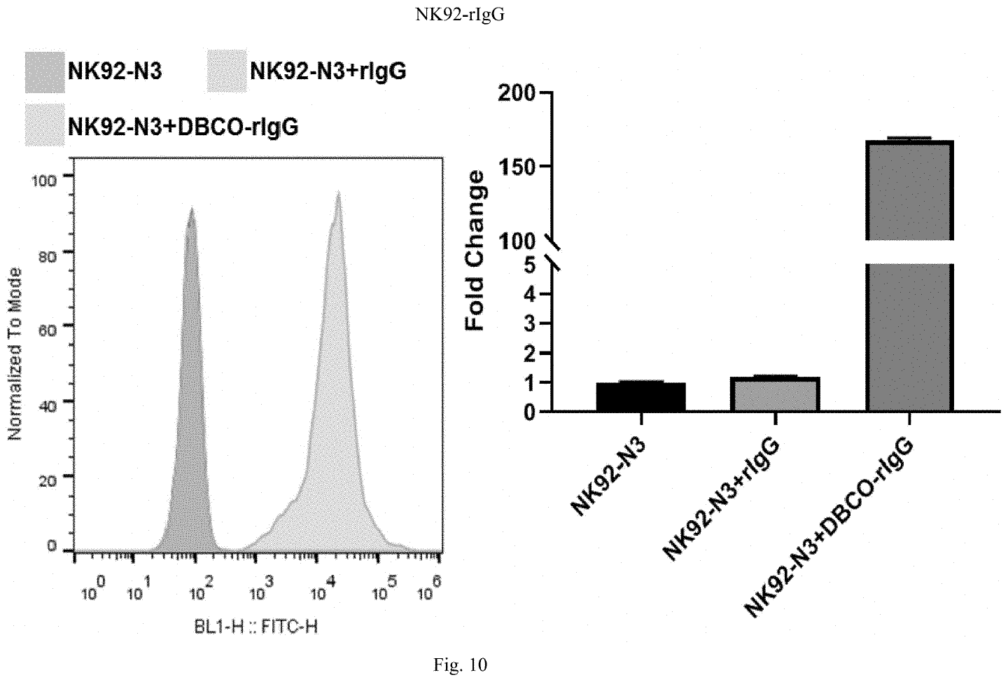

Three species of antibodies (hIgG, mIgG, rIgG) derived from human, mouse, and rabbit are respectively selected, and the antibodies are coupled to the surface of the NK92 cells according to the aforementioned coupling method. A corresponding secondary antibody having a fluorescent label is then used for fluorescent staining, and a fluorescent signal on the cell surface is detected by a flow cytometer. The experimental results are shown in FIGS. 8-10. Compared with a control group, the azide group on the sialic acid of the cell surface performs a bio-orthogonal reaction with a bio-orthogonal group of the conjugation compound of the antibody. A corresponding antibody fluorescence signal is successfully detected on the cell surface of the NK92 cells. The experimental results show that the technology of the present disclosure is also applicable for a coupling of the different species of the antibodies.

5.2 Coupling Multiple Species of the Antibodies Simultaneously on the Same Cell Surface

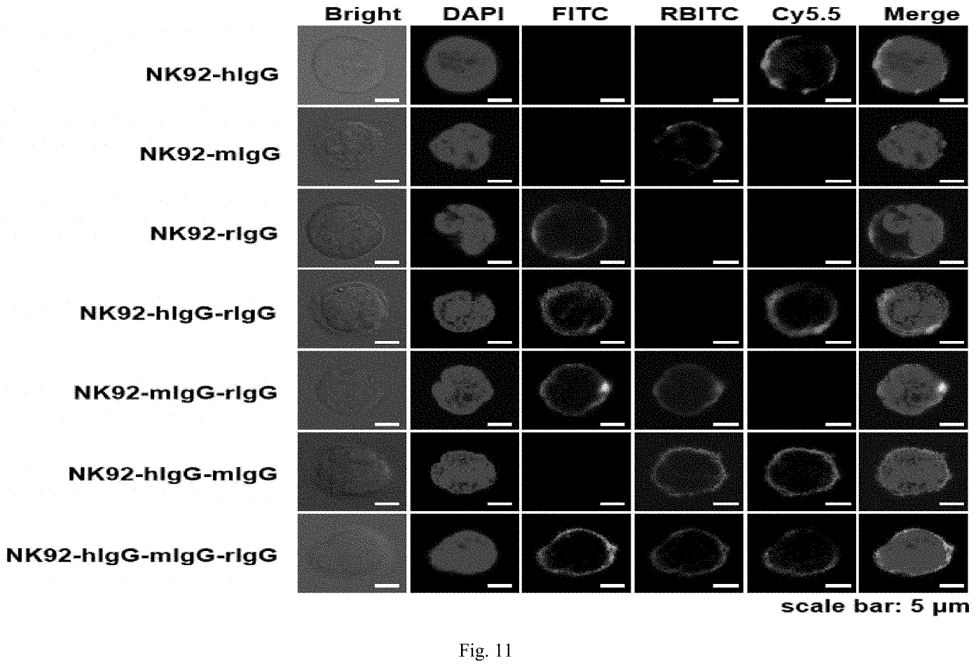

Another advantage of the technology of the cell surface coupled with the antibody of the present disclosure is that the multiple species of the antibodies can be simultaneously coupled to the same cell surface. In order to verify this conclusion, three species of the antibodies (hIgG, mIgG, rIgG) derived from human, mouse, and rabbit are respectively selected. In antibody-cell coupling steps, two (or three) species of the antibody prepared in advance are added to the NK92 cells with the surface modified with the azide group according to the aforementioned coupling method. After the coupling is complete, corresponding secondary antibodies with different fluorescent molecules are then used for fluorescent labeling. Finally, fluorescence signals on the cell surface are observed by a laser confocal microscopy. The experimental results are shown in FIG. 11: a fluorescent molecule labeled with the secondary antibody corresponding to the hIgG is Cy5.5, a fluorescent molecule labeled with the secondary antibody corresponding to mIgG is RBITC, and a fluorescent molecule labeled with the secondary antibody corresponding to rIgG is FITC. It can be seen from a confocal image that the corresponding fluorescent signals can be observed on the cell surface coupled with the multiple species of the antibodies, which indicates that the technology of the present disclosure realizes simultaneous coupling of the multiple species of the antibodies on the same cell surface.

5.3 Retention Time of the Antibodies on the Cell Surface

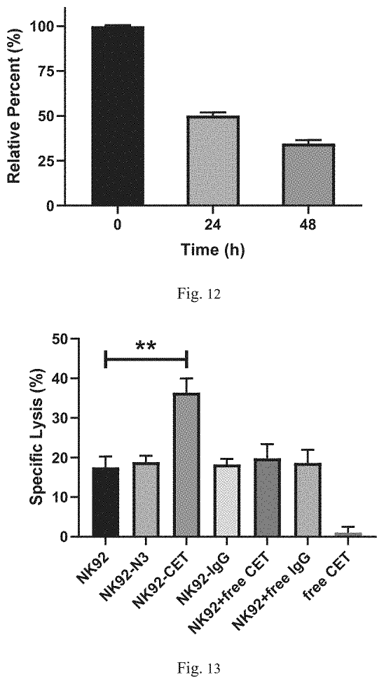

In order to detect a retention time of the antibodies coupled to the cell surface, cetuximab, which can inhibit a proliferation of human tumor cells expressing EGFR and induce apoptosis of human tumor cells, is coupled to the NK92 cells using the method of the present disclosure. Anti-Fc-APC is used to label the cetuximab on the cell surface, and fluorescence signals are detected by a flow cytometry. The fluorescence signals on the cell surface are then detected at different time points. The experimental results are shown in FIG. 12, and the antibody on the cell surface decreases by about 50% after 24 hours.

5.4 Killing Effects of the NK92 Cells Coupled with Cetuximab Against SW480 Cells In Vitro

As the cetuximab can inhibit the proliferation of the tumor cells expressing EGFR and induce apoptosis of the tumor cells, the SW480 cells are used to verify a killing ability of the NK92 cells coupled with the cetuximab (NK92-CET cells). The experimental results are shown in FIG. 13. As EGFR is expressed on a surface of the SW480 cells, the NK92 cells coupled with the cetuximab selectively bind to the SW480 cells through the antibodies on the surface of the NK92 cells to increase the ability for killing the tumor cells. It is consistent with the experimental results, a killing ability of the NK92-CET cells is nearly 2 times a killing ability of other control groups.

5.5 Antitumor Activities of the NK92-CET Cells in Mice In Vivo

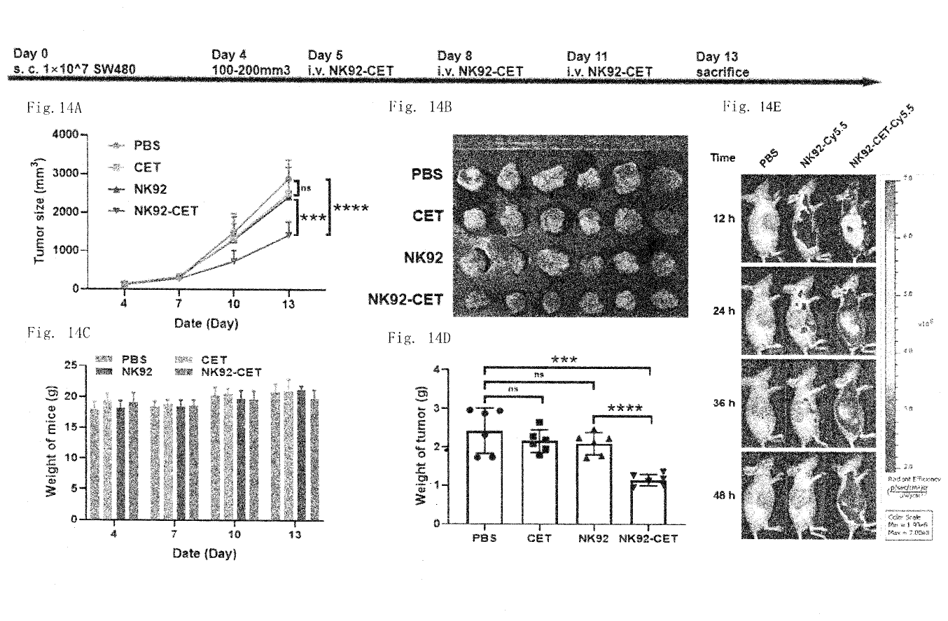

The SW480 cells are subcutaneously injected into nude mice to establish a model of a subcutaneous tumor. The NK92-CET cells are then respectively injected via a tail vein on day 5, 8 and 11, and growth states of the subcutaneous tumor are recorded (FIG. 14A). On day 13, the subcutaneous tumor is removed, and a weight of the subcutaneous tumor is measured (FIGS. 14B and 14D). During this period, body weights of mice are measured (as shown in FIG. 14C). In order to verify a tumor targeting activity of the NK92 cells coupled with the cetuximab in mice in vivo, a fluorescent molecule Cy5.5 is labeled on the NK92-CET cells for demonstrating a cell distribution in mice using in vivo imaging. The results are shown in FIG. 14E, which indicates that the NK92-CET cells have good tumor targeting in SW480 tumor mice. In summary, the experimental results show that in the technology of the present disclosure, specific antibodies are coupled to the cell surface to achieve selective targeting of tumor cells and improve the anti-tumor ability of the immune cells.

The aforementioned embodiments are merely some embodiments of the present disclosure, and the scope of the disclosure is not limited thereto. Thus, it is intended that the present disclosure cover any modifications and variations of the presently presented embodiments provided they are made without departing from the appended claims and the specification of the present disclosure by those skilled in the art.

What is claimed is:

1. A method for coupling an antibody to a surface of a cell, comprising:
(1) chemically modifying sialic acid to obtain a sialic acid derivative containing an azide group;
(2) absorbing the sialic acid derivative by the cell, and expressing the azide group on a membrane surface of the cell to obtain a cell modified with the azide group through a sialic acid metabolic pathway of the cell;
(3) modifying the antibody with a conjunction compound to obtain a modified antibody, wherein a first end of the conjunction compound has a first active group configured to react with a sulfhydryl group or an amino group, and a second end of the conjunction compound has a second active group configured to bio-orthogonally react with the azide group, and the first active group is connected to the sulfhydryl group or the amino group of the antibody by a first reaction; and
(4) co-culturing the modified antibody with the cell modified with the azide group to enable the second active group of the conjunction compound of the modified antibody to be connected to the azide group of the cell modified with the azide group by a second reaction.

2. The method according to claim 1, wherein the sialic acid derivative containing the azide group comprises a compound having any one of the following structural formulas:

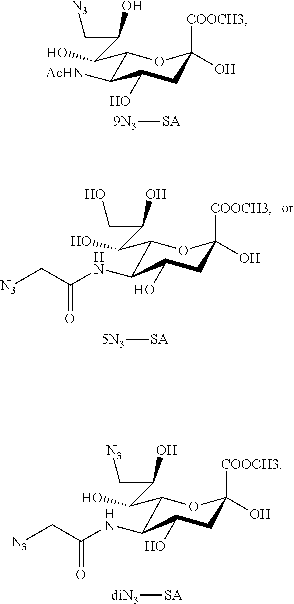

3. The method according to claim 1, wherein the first active group comprises a succinimide active ester group or a maleimide group.

4. The method according to claim 3, wherein the conjunction compound comprises a compound having any one of the following structural formulas:

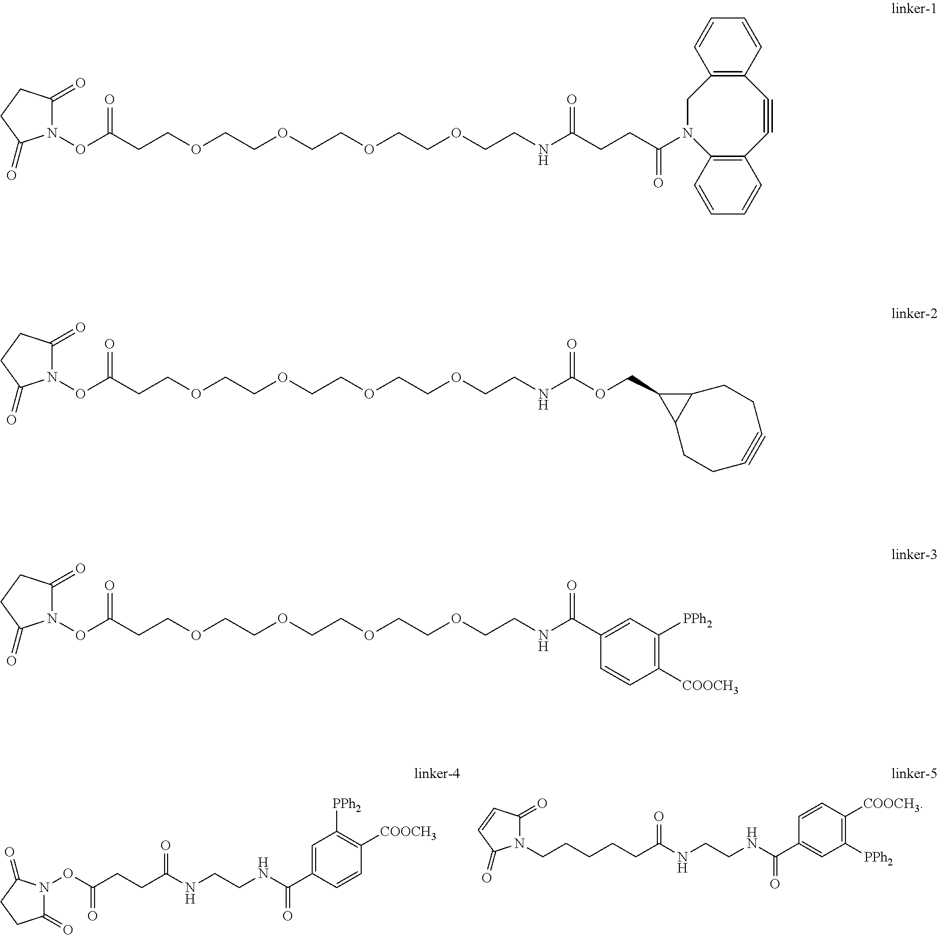

5. The method according to claim 1, wherein the cell is an immune cell.

6. A cell, wherein:
a surface of the cell comprises an azide group expressed on the surface by absorbing a sialic acid derivative containing the azide group through a sialic acid metabolic pathway,
the azide group is connected to an antibody through a conjunction compound,
a first end of the conjunction compound has a first active group configured to react with a sulfhydryl group or an amino group,
a second end of the conjunction compound has a second active group configured to react with the azide group by a bio-orthogonal reaction,
the first active group is connected to the sulfhydryl group or the amino group of the antibody by a first reaction, and
the second active group is connected to the azide group by a second reaction.

7. The cell according to claim 6, wherein the sialic acid derivative containing the azide group comprises a compound having any one of the following structural formulas:

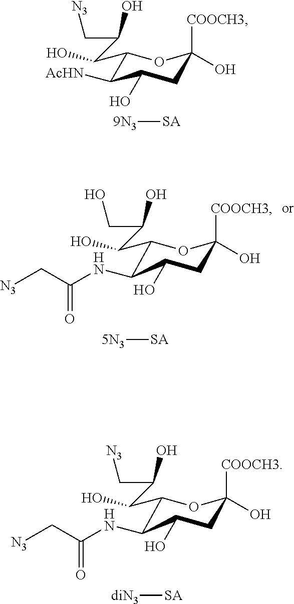

8. The cell according to claim 6, wherein the first active group comprises a succinimide active ester group or a maleimide group.

9. The cell according to claim 8, wherein the conjunction compound comprises a compound having any one of the following structural formulas:

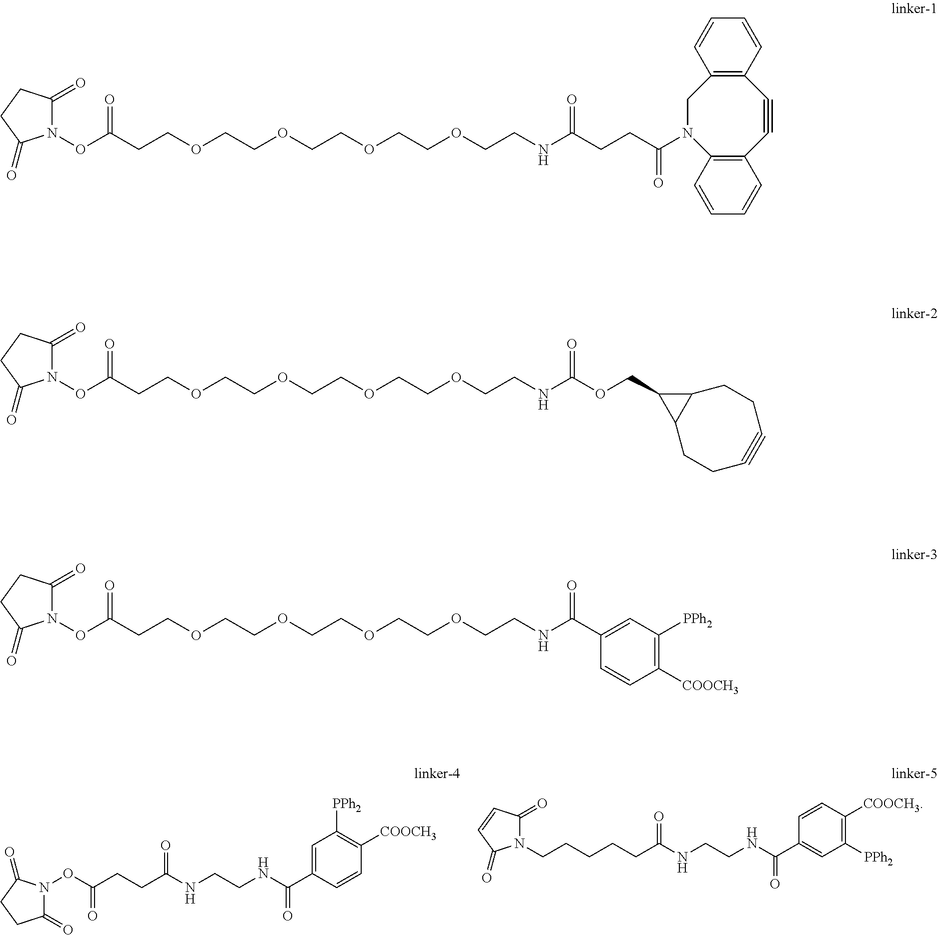

10. The cell according to claim 6, wherein the cell is an immune cell.

11. The cell according to claim 6, wherein different species of antibodies are coupled to 1 surface of a same cell.

12. A method for treating a disease of a target, comprising:
providing a pharmaceutical compound comprising an engineer cell and pharmaceutical acceptable additives, and
applying a therapeutic effective amount of the engineer cell to the target to treat the disease, wherein the engineer cell comprises an antibody coupled to a sialic acid derivative on a surface of the engineer cell.

13. The method according to claim 12, wherein the engineer cell is an immune cell.

14. The method according to claim 12, wherein the engineer cell is an original human T cell or a natural killer cell.

15. The method according to claim 12, wherein the surface of the engineer cell comprises an azide group expressed on the surface of the engineer cell by absorbing the sialic acid derivative comprising the azide group using a sialic acid metabolic pathway.

16. The method according to claim 1, wherein the cell is an original human T cell or a natural killer cell.

17. The cell according to claim 6, wherein the cell is an original human T cell or a natural killer cell.
